(19) United States
(12) Patent Application Publication
(10) Pub. No.: US 2007/0093524 A1
(43) Pub. Date: Apr. 26, 2007

(54) 5-Lipoxygenase modulators

(75) Inventors: Ponnal Nambi, Berwyn, PA (US); Liang Chen, Dayton, NJ (US); Elaine Quinet, Berwyn, PA (US); Jay Wrobel, Lawrenceville, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/544,073

(22) Filed: Oct. 6, 2006

Related U.S. Application Data
(60) Provisional application No. 60/729,914, filed on Oct. 25, 2005.

(51) Int. Cl. A61K 31/47

(52) U.S. Cl. 514/311; 514/312

(57) ABSTRACT

The present invention provides the use of Liver X Receptor (LXR) modulators that have been identified to downregulate 5-lipoxygenase gene expression in order to treat various diseases and disorders that involve the function of the 5-LO protein in intracellular signaling (or other cellular processes) or the function of protein products downstream of 5-LO in intracellular signaling (i.e., leukotrienes).

All patent applications, published patent applications, issued and granted patents, texts, and literature references cited in this specification are hereby incorporated herein by reference in their entirety.

### BACKGROUND OF THE INVENTION

The 5-lipoxygenase (5-LO or 5-LOX) pathway is the major source of proinflammatory leukotrienes (LTs) produced from the metabolism of arachidonic acid (AA). Cytosolic phospholipase A₂ (cPLA₂) liberates arachidonic acid from membrane phospholipids. The arachidonic acid is presented by Five-Lipoxygenase-Activating Protein (FLAP) to 5-LO. The 5-LO enzyme then coverts arachidonic acid to an unstable intermediate called 5-hydroperoxyeicosatetraenoic acid (5-HPETE), which is then dehydrated by 5-LO to produce LTA₄, a pivotal intermediate in the biosynthesis of inflammatory and anaphylactic mediator. Depending upon cell-type, LTA₄ is then converted into the chemoattractant LTB₄ (in neutrophils and monocytes), or LTC₄ (in human eosinophils, mast cells and basophils), which becomes sequentially cleaved to form the extracellular metabolites LTD₄ and LTE₄. Further, omega-oxidation and subsequent β-oxidation from the methyl terminus of the LTE₄ is a major metabolic route for sulfidopeptide leukotrienes, which are known as cysteinyl leukotrienes (cysLTs) in humans.

Abnormal production of LTs contributes to a variety of diseases and disorders because LTs are very potent molecules that act through receptors at subnanomolar concentrations. LTC₄ and LTD₄ cause hypotension in humans by causing a significant reduction in coronary blood flow. LTC₄ and LTD₄ constrict coronary arteries and distal segments of the pulmonary artery. LTC4 and LTD4 can cause plasma exudation and are more than 1000-times more potent than histamine in this respect. In addition to their effects on coronary blood flow, LTC4 and LTD4 are potent constrictors of bronchial smooth muscles. Leukotrienes also stimulate bronchial mucus secretion and cause mucosal edema. The sulfidopeptide leukotrienes have potent effects on microvasculature. Studies of mucosal biopsies from the bronchi of aspirin-intolerant asthmatics demonstrate that LTC4S is amplified, which correlates with an overproduction of cysLTs and bronchial hyperreactivity.

LTC4 and LTD4 show activity in relation to blood vessels and the bronchi, but they do not activate most leukocytes. However, LTB4 is unique in respect to other leukotrienes in that it acts as a potent chemotactic and chemokinetic lipid for peripheral mononuclear (PMN) leukocytes, eosinophils and monocytes (and macrophages). Normally, LTB₄ is biologically important for the removal of pathogens by activating and recruiting granulocytes to inflamed lesions as well as stimulating phagocytosis and the killing of microbes. However, higher concentrations of LTB₄ are responsible for causing PMN aggregation, degranulation and generation of superoxide, adhesion of neutrophils to vascular endothelium, and neutrophil trans-endothelial migration. Thus, an overproduction of LTB₄ can lead to various inflammatory diseases including psoriasis, bronchial asthma, rheumatoid arthritis, ulcerative colitis, and ischemic reperfusion injury in tissue.

Further, atherosclerosis is initiated by the trapping and oxidation of low-density lipoproteins (LDL) in the subendothelial layer of the artery wall, resulting in the stimulation of vascular cells to produce inflammatory molecules, including leukotrienes. This signals a cascade of leukocyte recruitment, further lipoprotein oxidation, foam cell formation, necrosis and fibroproliferation. Showing the relationship between 5-LO, leukotriene synthesis and atherosclerosis, 5-LO knockout mice show a dramatic decrease in aortic lesion development.

5-LO is also expressed and enzymatically active in various compartments of the mammalian brain, including central nervous system (CNS) neurons. Research into the CNS 5-LO pathway indicates that 5-LO may participate in a number of brain pathologies, including developmental neurometabolic diseases, stroke, seizures, Alzheimer's disease, aging-associated neurodegeneration, prion disease, multiple sclerosis, and brain tumors. Physiologically, 5-LO also appears to be involved in neurogenesis. Related to its role in cell proliferation, 5-LO also appears to be involved in the progression of cancer or neoplasia.

### SUMMARY OF THE INVENTION

The present invention provides the use of Liver X Receptor (LXR) modulators that have been identified to downregulate 5-lipoxygenase gene expression in order to treat various diseases and disorders that involve the function of the 5-LO protein in intracellular signaling (or other cellular processes) or the function of protein products downstream of 5-LO in intracellular signaling (i.e., leukotrienes). The invention provides LXR modulators having the Formula I: 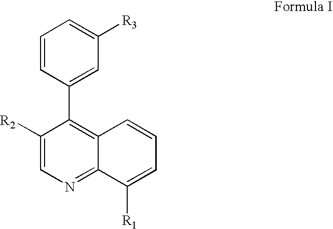
wherein R₁, R₂, and R₃ are as set forth below. Exemplary compounds of Formula I that can be used in the present methods are also described in the Examples. The compounds of the invention are herein referred to as “5-LO modulator compounds,” “5-LO modulating compounds” or “5-LO modulators.”

The invention also provides methods for identifying compounds, including LXR and PPAR (Peroxisome Proliferator-Activated Receptor) modulators, that can downregulate 5-LO gene expression. Further, the invention provides methods for monitoring the effectiveness of a compound in a subject by assaying for changes in 5-LO gene expression in particular tissues or cell-types.

In one aspect, the invention provides a method for downregulating 5-lipoxygenase gene expression comprising contacting a cell or a tissue with a compound of Formula I or a pharmaceutically acceptable salt thereof. The cell or tissue can comprise, for example, a platelet, a myeloid cell, a leukocyte, a neutrophil, a granulocyte, an eosinophil, a natural killer cell, a T-cell, a B-cell, a dendritic cell, an epidermal cell, a Langerhans cell, a keratinocyte, a glial cell, a macrophage, a monocyte, a mast cell, a pulmonary artery endothelial cell, an intestinal epithelial cell, vascular tissue, neural tissue, lung tissue, heart tissue, cardiovascular tissue, aorta tissue, coronary artery tissue, carotid artery tissue, renal tissue, pineal gland tissue, cerebral cortex tissue, hippocampus tissue, cerebellum tissue, ischemic flap tissue, pancreatic tissue or tumor tissue.

In another aspect, the invention provides a method for treating a condition, disease or disorder involving leukotriene-mediated inflammation or leukotriene-mediated cell signaling in a subject, the method comprising administering to the subject an effective amount of a compound of Formula I or a pharmaceutically acceptable salt thereof. The subject can be in need of treatment for a disease, disorder or condition comprising atherosclerosis, atherosclerotic lesions, high LDL cholesterol levels, low HDL cholesterol levels, abnormal reverse cholesterol transport, abnormal cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer or thyroiditis. In one aspect, the subject can be in need of treatment for a disease, disorder or condition comprising vascular dysfunction, hypertension, acute coronary syndrome, disorders of triglyceride metabolism, metabolic syndromes, Syndrome X, disorders in leukotriene synthesis, asthma, Sjogren-Larsson syndrome (SLS), stroke, seizure, prion disease, aging-associated neurodegeneration, or cancer.

In one aspect, the invention provides a method for reducing leukotriene synthesis in a subject, the method comprising administering to the subject an effective amount of a compound of Formula I or a pharmaceutically acceptable salt thereof.

In another aspect, the invention provides a method for treating inflammation in a subject, the method comprising administering to the subject an effective amount of a compound of Formula I or a pharmaceutically acceptable salt thereof.

In another aspect, the invention provides a method for treating atherosclerosis in a subject, the method comprising administering to the subject an effective amount of a compound of Formula I or a pharmaceutically acceptable salt thereof.

In another aspect, the invention provides a method for treating conditions, diseases or disorders involving leukotriene mediated intracellular or transcellular cell signaling in a subject, the method comprising administering to the subject an effective amount of a compound of Formula I or a pharmaceutically acceptable salt thereof.

In another aspect, the invention provides a method for treating conditions, diseases or disorders involving dysregulated 5-lipoxygenase-dependent cell signaling in a subject, the method comprising administering to the subject an effective amount of a compound of Formula I or a pharmaceutically acceptable salt thereof.

In another aspect, the invention provides a method for treating conditions, diseases or disorders involving 5-lipoxygenase-dependent lipid oxidation in a subject, the method comprising administering to the subject an effective amount of a compound of Formula I or a pharmaceutically acceptable salt thereof.

In one aspect, the invention provides a method for screening a compound to be a candidate for treating conditions, diseases or disorders involving leukotriene-mediated inflammation or leukotriene-mediated cell signaling (either intracellular or transcellular or both) (or other conditions, diseases or disorders that involves the function of 5-LO or its downstream products), the method comprising: (a) contacting a cell with the compound; and (b) determining whether 5-lipoxygenase gene expression is decreased in the cell of step (a) as compared to a cell that has not been contacted with the compound, wherein if 5-lipoxygenase gene expression is decreased in the cell of step (a), then the compound is a candidate for treating conditions, diseases or disorders involving leukotriene-mediated inflammation or leukotriene-mediated cell signaling. In a variation of this aspect, the cell can be transfected (transiently or stably) with a reporter gene expression construct comprising a 5-lipoxygenase cis regulatory element(s) that controls the expression of the reporter gene. In this variation, the compound is a candidate for treating conditions, diseases or disorders if the compound causes a reduction in the expression level of the reporter gene.

In one aspect, the invention provides a method for screening a compound to be a candidate for treating conditions, diseases or disorders involving leukotriene-mediated inflammation or 5-lipoxygenase mediated lipid oxidation (or other conditions, diseases or disorders that involves the function of 5-LO or its downstream products), the method comprising: (a) activating a macrophage cell with acetylated-LDL; (b) contacting the macrophage cell with the compound; (c) determining whether 5-lipoxygenase gene expression is decreased in the macrophage cell of step (b) as compared to a macrophage cell that has not been contacted with the compound, wherein if 5-lipoxygenase gene expression is decreased in the macrophage cell of step (b), then the compound is a candidate for downregulating 5-lipoxygenase gene expression. In a variation of this aspect, the cell can be transfected (transiently or stably) with a reporter gene expression construct comprising a 5-lipoxygenase cis regulatory element(s) that controls the expression of the reporter gene. In another variation of this aspect, a reporter gene can be targeted to integrate into the genome of the cell downstream of the 5-lipoxygenase reporter, such that the expression of the reporter gene is under control of the endogenous 5-lipoxygenase cis regulatory elements. In these variations, the compound is a candidate for treating conditions, diseases or disorders if the compound causes a reduction in the expression level of the reporter gene.

In the methods for screening a compound to be a candidate for treating conditions, diseases or disorders contemplated by the invention, the compound to be screened can be a Liver X Receptor (LXR) modulator compound, or a Peroxisome Proliferator-Activated Receptor (PPAR) modulator compound. In another aspect, the compound to be screened comprises a quinoline compound.

In the methods for screening a compound to be a candidate for treating conditions, diseases or disorders, exemplary conditions, diseases or disorders can comprise atherosclerosis, atherosclerotic lesions, high LDL cholesterol levels, low HDL cholesterol levels, abnormal reverse cholesterol transport, abnormal cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer or thyroiditis.

In one aspect, the invention provides a method for assessing or testing the efficacy of a 5-LO modulator compound that has been administered to a subject, the method comprising (a) isolating a blood, tissue or other cellular sample from the subject before and after administration of the compound, and (b) determining whether 5-LO gene expression is reduced in the sample obtained after administration as compared to the sample obtained before administration. The sample obtained after administration can be after multiple administrations if the dosage regime of the compound involves repeated or multiple administrations.

In one aspect, the invention provides a kit that comprises a unit dosage form of a 5-LO modulator compound having the Formula I or a pharmaceutically acceptable salt thereof. The unit dosage form can comprise a container, which can be sterile, containing an effective amount of the 5-LO modulator compound and a physiologically acceptable carrier or vehicle. The kit can further comprise a label or printed instructions instructing the use of the 5-LO modulator compound to treat or prevent a condition. The kit can also further comprise a unit dosage form of another therapeutic agent, for example, a container containing an effective amount of the other therapeutic agent. In another aspect, the kit comprises a container containing an effective amount of a 5-LO modulator compound having the Formula I or a pharmaceutically acceptable salt thereof and an effective amount of another therapeutic agent. Kits of the invention can further comprise a device that is useful for administering the unit dosage forms. Examples of such a device include, but are not limited to, a syringe, a drip bag, a patch, an inhaler, and an enema bag.

### DETAILED DESCRIPTION OF THE INVENTION

LXRs, originally identified from liver as orphan receptors, are members of the nuclear hormone receptor super family and are involved in the regulation of cholesterol and lipid metabolism. They are ligand-activated transcription factors and bind to DNA as obligate heterodimers with retinoid X receptors. Activation of LXRs by oxysterols (endogenous ligands) in macrophages results in the expression of several genes involved in lipid metabolism and reverse cholesterol transport including ABCA1, ABCG1 and ApoE. Activation of LXRs also results in the inhibition of inflammation and proinflammatory gene expression in three different models of inflammation (LPS-induced sepsis, acute contact dermatitis of the ear and chronic atherosclerotic inflammation of the artery wall).

Because activation of LXRs can result in the inhibition of inflammation and proinflammatory gene expression, LXR modulating compounds (see U.S. Patent Application Publication No. US 2005-0131014 A1) and PPAR modulating compounds were examined for their ability to downregulate 5-LO gene expression. From these studies, a specific subclass of LXR/PPAR modulating compounds was identified to downregulate 5-LO gene expression (see Example 9), and these compounds are herein referred to as 5-LO modulator compounds.

### Definitions

The terms “administer”, “administering”, or “administration”, as used herein refer to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “subject” as used herein includes, without limitation, a human, mouse, rat, guinea pig, dog, cat, horse, cow, pig, monkey, chimpanzee, baboon, or rhesus. In one embodiment, the subject is a mammal. In another embodiment, the subject is a human.

The term “pharmaceutically acceptable salt” as used herein refers to a salt of an acid and a basic nitrogen atom of a compound of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes a hydrate of a compound of the present invention.

The following abbreviations are used herein: “ESI” means electrospray ionization; “HRMS” means high-resolution mass spectrometry; “MS” means mass spectrometry; “m/z” means the mass-to-charge ratio of an atomic or molecular ion.

### Compounds of the Invention

In one embodiment, the invention provides 5-LO modulator compounds having the Formula I: 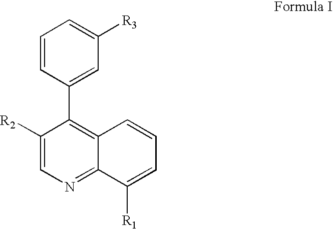
and pharmaceutically acceptable salts thereof,
wherein
R₁ is —CF₃ or —Cl;
R₂ is —CH₃,

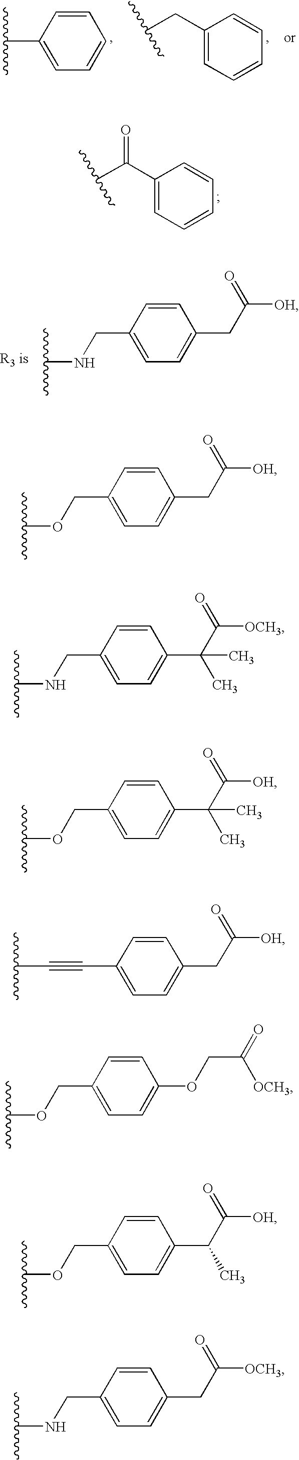

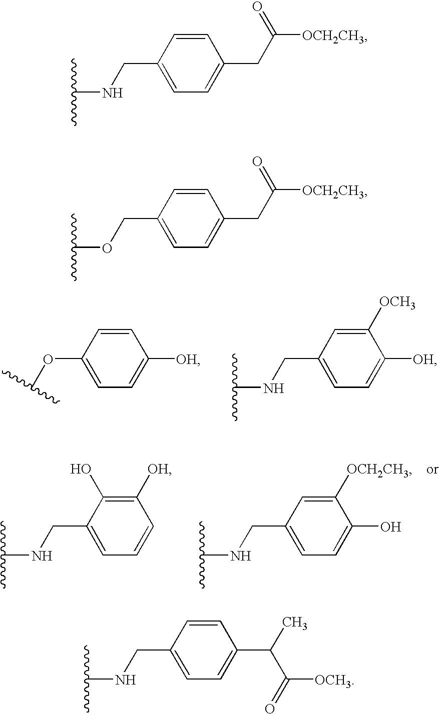

In one embodiment, R₁ is —CF₃.

In another embodiment, R₂ is

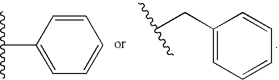

In another embodiment, R₃ is

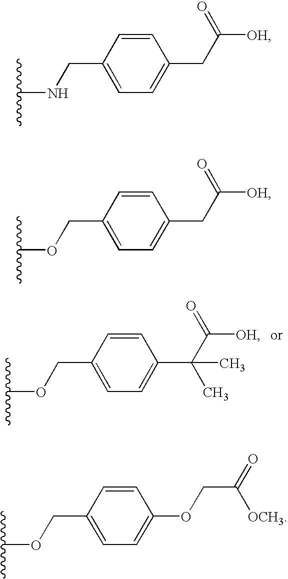

In other embodiments, the 5-LO modulator compound is: (4-{[3-(3-Benzyl-8-trifluoromethyl-quinolin-4-yl)-phenylamino]-methyl}-phenyl)-acetic acid (Compound I-1); {4-[3-(3-Benzyl-8-trifluoromethyl-quinolin-4-yl)-phenoxymethyl]-phenyl}-acetic acid (Compound I-2); {4-[3-(3-Phenyl-8-trifluoromethyl-quinolin-4-yl)-phenoxymethyl]-phenyl}-acetic acid (Compound I-3); {4-[3-(3-Methyl-8-trifluoromethyl-quinolin-4-yl)-phenoxymethyl]-phenyl}-acetic acid (Compound I-4); {4-[3-(8-Chloro-3-phenyl-quinolin-4-yl)-phenoxymethyl]-phenyl}-acetic acid (Compound I-5); {4-[3-(8-Chloro-3-methyl-quinolin-4-yl)-phenoxymethyl]-phenyl}-acetic acid (Compound I-6); 2-{4-[3-(3-Benzyl-8-trifluoromethyl-quinolin-4-yl)-phenoxymethyl]-phenyl}-2-methyl-propionic acid (Compound I-7); 2-{4-[3-(8-Chloro-3-phenyl-quinolin-4-yl)-phenoxymethyl]-phenyl}-2-methyl-propionic acid (Compound I-8); {4-[3-(3-Benzyl-8-trifluoromethyl-quinolin-4-yl)-phenylethynyl]-phenyl}-acetic acid (Compound I-9); (2R)-2-{4-[3-(3-Methyl-8-trifluoromethyl-quinolin-4-yl)-phenoxymethyl]-phenyl}-propionic acid (Compound I-10); (4-{[3-(3-Benzoyl-8-trifluoromethyl-quinolin-4-yl)-phenylamino]-methyl}-phenyl)-acetic acid methyl ester (Compound I-11); (4-{[3-(3-Benzyl-8-trifluoromethyl-quinolin-4-yl)-phenylamino]-methyl}-phenyl)-acetic acid hydrazide (Compound I-12); (3-{[3-(3-Benzoyl-8-trifluoromethyl-quinolin-4-yl)-phenylamino]-methyl}-phenyl)-acetic acid ethyl ester (Compound I-13); {4-[3-(3-Phenyl-8-trifluoromethyl-quinolin-4-yl)-phenoxymethyl]-phenyl}-acetic acid ethyl ester (Compound I-14); {4-[3-(3-Benzyl-8-trifluoromethyl-quinolin-4-yl)-phenoxymethyl]-phenyl}-acetic acid ethyl ester (Compound I-15); 4-[3-(3-Benzyl-8-trifluoromethyl-quinolin-4-yl)-phenoxy]-phenol (Compound I-16); 4-{[3-(3-Benzyl-8-trifluoromethyl-quinolin-4-yl)-phenylamino]-methyl}-2-methoxy-phenol (Compound I-17); 3-{[3-(3-Benzyl-8-trifluoromethyl-quinolin-4-yl)-phenylamino]-methyl}-benzene-1,2-diol (Compound I-18); 4-{[3-(3-Benzyl-8-trifluoromethyl-quinolin-4-yl)-phenylamino]-methyl}-2-ethoxy-phenol (Compound I-19); (4-{[3-(3-Methyl-8-trifluoromethyl-quinolin-4-yl)-phenylamino]-methyl}-phenyl)-acetic acid methyl ester (Compound I-20); 2-(4-{[3-(3-Methyl-8-trifluoromethyl-quinolin-4-yl)-phenylamino]-methyl}-phenyl)-propionic acid methyl ester (Compound I-21); 2-Methyl-2-(4-{[3-(3-methyl-8-trifluoromethyl-quinolin-4-yl)-phenylamino]-methyl}-phenyl)-propionic acid methyl ester (Compound I-22); {4-[3-(3-Phenyl-8-trifluoromethyl-quinolin-4-yl)-phenoxymethyl]-phenoxy}-acetic acid methyl ester (Compound I-23); or a pharmaceutically acceptable salt thereof.

In another embodiment, the invention provides methods of making the 5-LO modulator compounds of the present invention.

The 5-LO modulator compounds of the present invention can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

The compounds and pharmaceutically acceptable salts of compounds of the present invention can be made generally according to the synthetic procedures outlined in U.S. Patent Application Publication No. US 2005-0131014 A1 or by Schemes 1-5 below.

For example, according to Scheme 1 shown below, aniline (1) can be condensed with diethyl ethoxymethylenemalonate (2) to provide compound (3). Compound (3) is cyclized thermally to provide the quinoline of formula (4). Conversion of the phenol of (4) to the chloride of formula (5) can be accomplished readily with chlorinating agents such as phosphorus oxychloride. Reaction of the ester moiety of (5) with an organolithium reagent (Ph₂Li) provides the compound of formula (6). Reaction of (6) with a boronic acid reagent m-R₃-phenyl-(OH)₂ in the presence of a palladium catalyst provides the compound (7).

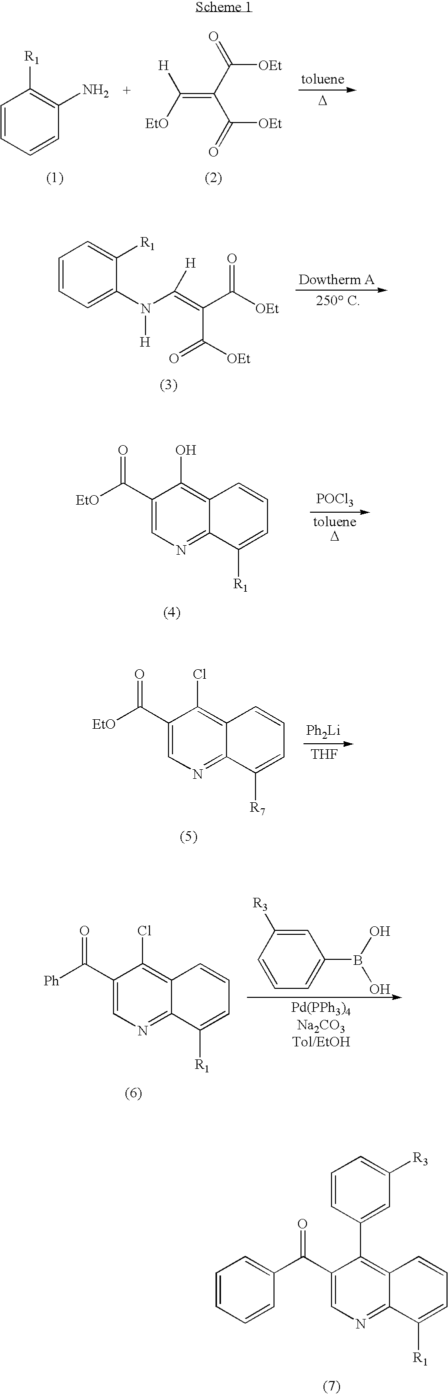

According to Scheme 2 shown below, the carbonyl group of the compound (7), e.g., compounds of Formula I, where R₂ is —C(O)Ph, can be reduced to provide the compound (8), e.g., compounds of Formula I wherein R₂ is —CH₂-Ph. Treatment of compound (7) with hydrazine followed by potassium hydroxide provides compound (8).

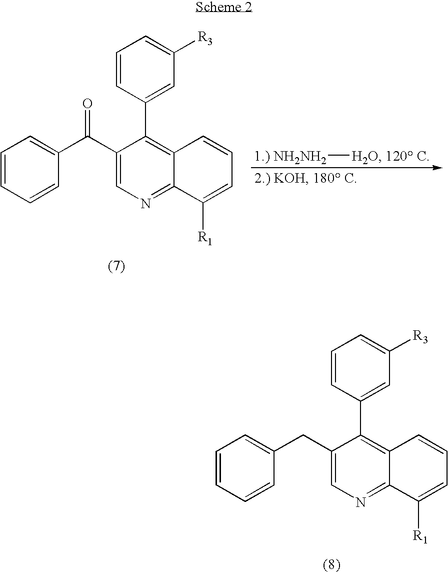

According to Scheme 3 shown below, the compounds of Formula I where R₂ is —CH₃ or -Ph can also be prepared. The compound (11) is converted to the N-methyl, N-methoxy amide (“Weinreb amide”) compound (12) under standard amidation conditions. Reaction of the amide (12) with a lithio or Grignard reagent of formula R₃Li or R₃MgBr at low temperature provides the compound (13). Alternatively, the compound (9) is lithiated alpha to the fluorine atom and then treated with an appropriately substituted aldehyde. The resulting alcohol compound (10) is converted to the ketone compound (13) under standard oxidation conditions. Conversion of compound (13) into the aniline is accomplished with ammonium hydroxide at elevated temperature. Substituted aniline compound (14) undergoes clean condensation, cyclization in acetic acid with a catalytic amount of sulfuric acid at elevated temperature to provide the compound (15).

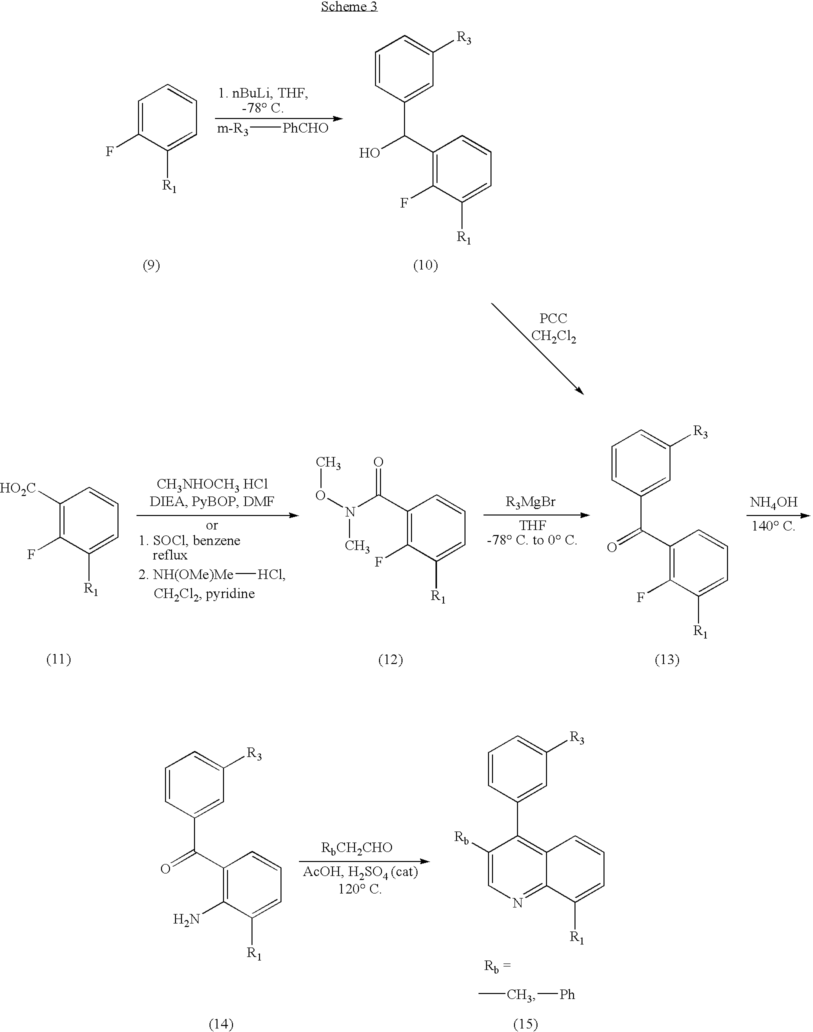

According to Scheme 4 shown below, certain compounds of Formula I prepared by Schemes 1-3, contain a (CH₂)ⱼOH moiety on the phenyl ring that is attached to the 4-position of the quinoline ring system. Alkylation of the —OH of compound (16) with an alkylating agent RX′ using potassium, sodium or cesium carbonate as the base provides the alkylated compound (17). Alternatively, if j is 1 or more and ROH is a phenol or substituted phenol, or j is 0 and ROH is an alcohol where the OH is connected to a sp³ hybridized carbon, then the alcohol of compound (16) and the ROH can be reacted with triphenylphosphine (PPh₃) and diisopropylazodicarboxylate (DIAD) to form the ether of compound (17). Alternatively, arylation of the —OH of compound (16), when j=0, with an aryl iodide, bromide or boronic acid using an appropriate copper catalyst, and a tertiary amine base if necessary provides the aryl ether of compound (17). If the R group of compound (17) contains a carboxylic acid ester moiety, this moiety can be transformed to the carboxylic acid upon treatment with aqueous lithium, sodium or potassium hydroxide in a suitable organic solvent.

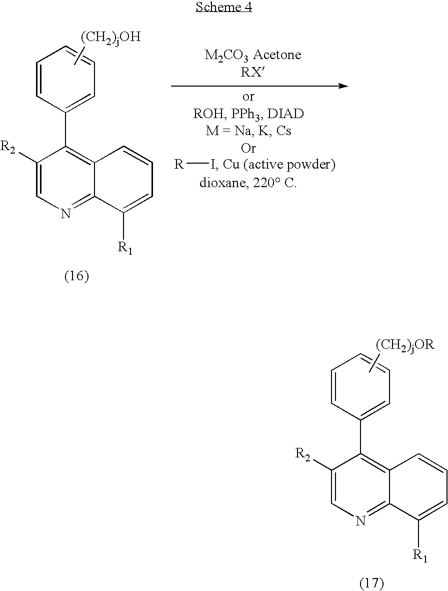

According to Scheme 5 provided below, certain compounds of Formula I prepared by Scheme 1-3, contain a free NH₂ moiety on the phenyl ring that is attached to the 4-position of the quinoline ring system. Treatment of the free amine of compound (18) with an aldehyde (RCHO) and a reducing agent such as NaBH(OAc)₃, results in the secondary amine compound (19). Compound (19) can also be obtained upon treating the starting primary amine with an alkylating agent (RX′), where X′ is a leaving group, in the presence of a base. If the R group of compound (19) contains a carboxylic acid ester moiety this moiety can be transformed to the carboxylic acid upon treatment with aqueous lithium, sodium or potassium hydroxide in a suitable organic solvent.

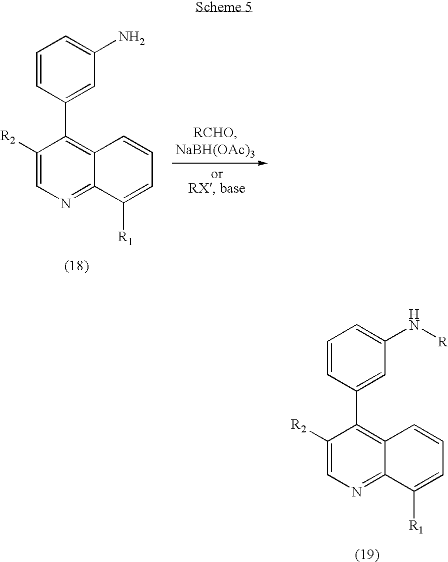

One of skill in the art will recognize that Schemes 1-5 can be adapted to produce the compounds and pharmaceutically acceptable salts of compounds according to the present invention.

### Methods of Treatment

LO is a protein with catalytic activity that is important for transforming arachidonic acid into leukotrienes. Leukotrienes are lipid messengers that play central roles in immune response, including inflammatory responses, and tissue homeostasis. Leukotrienes may also be involved in neurogenesis and neurological disease as both intracellular second messengers and as transcellular mediators. 5-LO also has the ability to bind and affect the function of a number of cellular proteins, including cytoskeletal proteins, the dicer protein (which is an enzyme critical for the process of RNA interference) and the Trk receptor. Therefore, 5-LO can be an attractive target for a number of conditions, disorders or diseases that involve inflammation, leukotriene intracellular and/or transcellular signaling, or cell proliferation (including neoplastic transformation).

Therefore, the invention provides methods for treating disorders by downregulating 5-LO gene expression. In one embodiment, the disorders involve inflammatory activities mediated by leukotrienes. In another embodiment, the disorders can involve cell proliferation due to leukotriene signaling. The downregulation of 5-LO gene expression can be accomplished by administering to a subject an effective amount of a 5-LO modulator compound, the compound the Formula I or a pharmaceutically acceptable salt thereof.

Specific conditions, diseases or disorders that can be treated or inhibited by the compounds of the invention include, but are not limited to, atherosclerosis, atherosclerotic lesions, lowering LDL cholesterol levels, increasing HDL cholesterol levels, increasing reverse cholesterol transport, inhibiting cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, treatment or inhibition of Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer and thyroiditis.

The compounds of the invention can also be used in methods for inhibiting or reducing 5-LO gene expression. These methods also contemplate inhibiting or reducing 5-LO gene expression in specific tissues or cell types, including but not limited to, platelets, myeloid cells, leukocytes, neutrophils, granulocytes, eosinophils, natural killer cells, T-cells, B-cells, dendritic cells, epidermal cells, Langerhans cells, keratinocytes, glial cells, macrophages, monocytes, mast cells, pulmonary artery endothelial cells, intestinal epithelial cells, vascular tissue, neural tissue, lung tissue, heart tissue, cardiovascular tissue, aorta tissue, coronary artery tissue, carotid artery tissue, renal tissue, pineal gland tissue, cerebral cortex tissue, hippocampus tissue, cerebellum tissue, ischemic flap tissue, and tumor tissue.

### Therapeutic/Prophylactic Administration

When administered to a subject, the 5-LO modulators can be administered as a component of a composition that comprises a physiologically acceptable carrier or vehicle. The present compositions, which comprise a 5-LO modulator compound having the Formula I, can be administered orally. The compounds of the invention can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the 5-LO modulator compounds into the bloodstream. The mode of administration is left to the discretion of the practitioner.

In one embodiment, the 5-LO modulating compounds are administered orally. In another embodiment,.the 5-LO modulators are administered intravenously. In other embodiments, it can be desirable to administer the 5-LO modulator compounds locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the 5-LO modulator compounds into the cardiovascular system, pulmonary system, lymphatic system, central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the 5-LO modulator compounds can be formulated as a suppository, with traditional binders and excipients such as triglycerides.

In another embodiment the 5-LO modulator compounds can be delivered in a vesicle, in particular a liposome (see Langer, Science 249:1527-1533 (1990)).

In yet another embodiment the 5-LO modulator compounds can be delivered in a controlled-release system or sustained release system (see, e.g., Goodson, in Medical Applications of Controlled Release, CRC Press, Boca Raton, Fla., vol. 2, pp. 115-138 (1984)). Other controlled or sustained release systems discussed in the review by Langer (Science 249:1527-1533 (1990)) can be used. In one embodiment a pump can be used (Langer, Science 249:1527-1533 (1990); Sefton, CRC Crit. Ref. Biomed. Eng. 14:201 (19.87); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1985); During et al., Ann. Neural. 25:351 (1989); and Howard et al., J. Neurosurg. 71:105 (1989)).

In yet another embodiment a controlled or sustained release system can be placed in proximity of a target of the 5-LO modulator compounds, e.g., the heart, spinal column, brain, skin, lung, or gastrointestinal tract, thus requiring only a fraction of the systemic dose. In one embodiment, the controlled or sustained release system comprises a stent that is coated with a 5-LO modulator compound of the invention.

The present compositions can optionally comprise a suitable amount of a physiologically acceptable excipient so as to provide the form for proper administration to the subject.

Such physiologically acceptable excipients can be liquids, such as water and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water can be a useful excipient when the 5-LO modulator compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills; pellets, capsules, capsules containing liquids, powders, sustained release formulations, suppositories, emulsions, aerosols, sprays, suspensions, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment, the 5-LO modulator compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a 5-LO modulator compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the 5-LO modulator compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the 5-LO modulator compounds are to be administered by infusion, they can be dispensed, for example, with an infusion bottle containing sterile pharmaceutical grade water or saline. Where the 5-LO modulator compounds are administered by injection, an ampule of sterile water for injection or saline can be provided so that the ingredients can be mixed prior to administration.

The 5-LO modulator compounds can be administered by controlled-release or sustained release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled or sustained release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled or sustained release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled or sustained release.

In one embodiment a controlled or sustained release composition comprises a minimal amount of a 5-LO modulator compound to reduce inflammation in a minimal amount of time. Advantages of controlled or sustained release compositions include extended activity of the 5-LO modulator compound, reduced dosage frequency, and increased subject compliance. In addition, controlled or sustained release compositions can favorably affect the time of onset of action or other characteristics, such as blood levels of the 5-LO modulator compound, and can thus reduce the occurrence of adverse side effects.

Controlled or sustained release compositions can initially release an amount of a 5-LO modulator compound that promptly produces the desired therapeutic or prophylactic effect, and gradually and continually release other amounts of the 5-LO modulator to maintain this level of therapeutic or prophylactic effect over an extended period of time. To maintain a constant level of the 5-LO modulator in the body, the 5-LO modulator can be released from the dosage form at a rate that will replace the amount of 5-LO modulator being metabolized and excreted from the body. Controlled or sustained release of an active ingredient can be stimulated by various conditions, including but not limited to, changes in pH, changes in temperature, concentration or availability of enzymes, concentration or availability of water, or other physiological conditions or compounds.

The amount of the 5-LO modulator that is effective: (i) in the treatment or prevention of a condition; (ii) to temporarily reduce inflammation; or (iii) to reduce the severity or amount of atherosclerotic plaques in a subject, can be determined by standard clinical techniques. In addition, in vitro or in vivo assays can optionally be employed to help identify optimal dosage ranges. The precise dose to be employed will also depend on the route of administration, and the seriousness of the condition being treated and should be decided according to the judgment of the practitioner and each subject's circumstances in view of, e.g., published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one 5-LO modulator compound is administered, the effective dosage amounts correspond to the total amount administered.

The 5-LO modulator compounds can be assayed in vitro or in vivo for the desired therapeutic or prophylactic activity prior to use in humans. Subject model systems can be used to demonstrate safety and efficacy.

The present methods for treating or preventing a condition in a subject in need thereof can further comprise administering another therapeutic agent to the subject being administered a 5-LO modulator compound. In one embodiment the other therapeutic agent is administered in an effective amount. In one embodiment, the other therapeutic agent is a LXR modulator or PPAR modulator that is not also a 5-LO modulator. In another embodiment, the other therapeutic agent can be an agent useful for reducing any potential side effect of a 5-LO modulator compound.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the 5-LO modulator compound is less than its effective amount would be where the other therapeutic agent is not administered. In this case, without being bound by theory, it is believed that the 5-LO modulator compounds and the other therapeutic agent act synergistically to treat or prevent a condition.

### Kits

The invention encompasses kits that can simplify the administration of a 5-LO modulator compound to a subject.

A typical kit of the invention comprises a unit dosage form of a 5-LO modulator compound. In one embodiment, the unit dosage form is a container, which can be sterile, containing an effective amount of a 5-LO modulator compound and a physiologically acceptable carrier or vehicle. The kit can further comprise a label or printed instructions instructing the use of the 5-LO modulator compound to treat or prevent a condition. The kit can also further comprise a unit dosage form of another therapeutic agent, for example, a container containing an effective amount of the other therapeutic agent. In one embodiment, the kit comprises a container containing an effective amount of a 5-LO modulator compound and an effective amount of another therapeutic agent. Examples of other therapeutic agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag.

### Methods for Identifying/Screening 5-LO Modulator Compounds

The invention provides methods for identifying or screening compounds as candidates for the treatment or inhibition of diseases, disorder or conditions relating to 5-LO, which include, but are not limited to, atherosclerosis, atherosclerotic lesions, lowering LDL cholesterol levels, increasing HDL cholesterol levels, increasing reverse cholesterol transport, inhibiting cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, treatment or inhibition of Alzheimer's disease, multiple sclerosis, restenosis, inflammatory bowel disease (IBD), Crohn's disease, endometriosis, celiac, cancer, lung cancer and thyroiditis, that can downregulate or alter 5-LO gene expression.

The methods for identifying or screening compounds are based upon the determination of whether a compound can affect 5-LO gene expression. The compounds that can be tested are compounds that may be useful in the treatment or inhibition of treatment or inhibition of LXR and/or PPAR mediated diseases. In one embodiment, the compounds can comprise a quinoline compound that is useful in the treatment or inhibition of LXR and/or PPAR mediated diseases. Contemplated quinoline compounds that can be tested include, but are not limited to, the compounds as described in U.S. Patent Application Publication No. US 2005-0131014 A1.

Generally, the candidate compound is added to a cell-line that expresses the 5-LO gene. Cell-lines that naturally express the 5-LO gene are preferred. Cell-lines that endogenously express the 5-LO gene are preferred because transient expression systems may not contain all of the necessary cis regulatory elements to recreate the in vivo regulation of 5-LO gene expression. However, transient expression system can be used where a cell-line that does not endogenously express 5-LO is transfected with an expression vector comprising the 5-LO gene under control of its native cis regulatory elements. The native cis regulatory elements can comprise, for example, the promoter (In, K. H. et al., J. Clin. Invest., 1997, 99, 1130; Silverman, E. S. et al., Am. J. Respir. Cell. Mol. Biol., 1998, 19, 316.), enhancers and silencers. The promoter can contain polymorphisms that may be present in specific sub-populations, including sub-populations based on race, predisposition to a disease or disorder, or based on having an allergic reaction to a drug. For example, examination of genomic DNA from asthmatic as well as non-asthmatic subjects revealed the occurrence of natural mutations within the promoter region (Silverman, E. S., Drazen, J. M. Am. J. Respir. Crit. Care Med., 2000, 161, S77).

In one embodiment, a macrophage cell line is used that naturally expresses the 5-LO gene is used. The macrophage cell line can be a human or a murine cell line. The human cell line can be, for example, the THP-1 cell line. The murine cell line can be, for example, the J774 cell line. Generally, for macrophage or monocyte cell lines, the cells are activated with acetylated-LDL. After activation, the cells are ready to be tested with candidate compounds. After cells are incubated with candidate compounds, total RNA can be prepared and 5-LO gene expression can be assessed by RT-PCR (see Example 9). If the amount of 5-LO mRNA is inhibited or reduced by the presence of a compound, then the compound is a candidate for treating the disorders, conditions and diseases mentioned herein.

In another embodiment, cell-lines that endogenously express 5-LO can be modified such that 5-LO gene expression can be assayed by reporter protein detection. For example, cell-lines can be manipulated such that a gene encoding a fluorescent protein is specifically targeted downstream of the 5-LO gene promoter. In this manner, the effect of a compound on 5-LO gene expression can be determined by assaying for a reduction in reporter gene expression. Gene-targeting methods can be conducted according to standard procedures known in the art.

In another embodiment, cell-lines that transiently express 5-LO can be modified such that the expression vector comprising 5-LO cis regulatory elements control the expression of a reporter gene encoding a reporter protein or a fusion protein comprising 5-LO and a reporter protein.

The reporter gene can encode a fluorescent protein or a luminescent protein, for example. The fluorescent proteins include, but are not limited to, red fluorescent protein (RFP), yellow fluorescent protein (YFP), cyan fluorescent protein (CFP), green fluorescent protein (GFP), blue fluorescent protein (BFP), sapphire fluorescent protein, and their variants. The luminescent proteins can include, for example, luciferase and alkaline phosphatase. With fluorescent proteins, the cells do not need to be manipulated in order to detect whether the compound affects 5-LO gene regulation because fluorescence can easily be detected in cell-based methods. Further, when luminescent proteins are used, then their substrates can be added directly to the supernatant of wells in order to assay for whether the luminescent proteins are produced and secreted by the cells. Fluorescence or luminescence can be detected by high-throughput methods by using microplate readers.

The present methods, including RT-PCR based methods, can be conducted in a high-throughput manner because cells can be grown in individual wells of multi-well plates, including microplates. Microplates can comprise polycarbonate material, and have 96-wells, 384 wells, or 1536 wells for example. Individual wells in the 96-well microplate often hold working volumes of about 250 μL. Many companies offer a variety of plate formats and membranes that allow a wide range of functional cell-based assay protocols to be performed. For example, Millipore (Billerica, Mass.) offers a variety of MultiScreen® plates that can be used for both cell culture growth and high-throughput assay testing. The MultiScreen-FL plates are designed for cell viability and fluorescent detection. The plates contain 96 individual wells, where 96 samples can be incubated, washed and assayed. Such plates allow fluorescent signal to be directly detected and quantitated in the plates without sample transfer. Such plates are also compatible with robotics, such as automated liquid handling systems.

With respect to certain embodiments, fluorescent or luminescent reporter activity in microplates can be detected and quantitated by microplate readers. For example, Molecular Devices (Sunnyvale, Calif.) offers the SpectraMax® M5 microplate reader, that is compatible with microplates having 6-384 wells. Detection modalities include absorbance (UV-Vis), fluorescence intensity (FI), fluorescence polarization (FP), time-resolved fluorescence (TRF) and luminescence (Lum), and therefore fluorescence from GFP can be detected and quantitated. With respect to robotics, Molecular Devices offers the SynchroMax ET Plate Handler that provides plate capacity of up to 120 microplates and can be expanded to 320 plates to create an integrated workstation that provides walk-away automation. ELISA or cell-based assays using absorbance, fluorescence and/or luminescence detection modes with dispensing and microplate cell washing can be automated to further increase the throughput and efficiency.

High-throughput screening of fluorescent-based methods can also be conducted by FACS (fluorescent activated cell sorter). Flow cytometry allows for rapid screening and sorting of individual cells based on fluorescence emission, including GFP emission. High-throughput screening, whether conducted by microplate or by FACS, can be conducted iteratively, where candidate a compound can be repeatedly tested in the same assay to determine whether same results are obtained. In this manner, false positives can be eliminated.

### Methods for Testing the Efficacy of 5-LO Modulators

In a related embodiment to the methods for identifying or screening compounds as potential candidates, the invention provides for methods of testing the efficacy of a compound for the treatment or inhibition of diseases, disorder or conditions relating to 5-LO, which include, but are not limited to, atherosclerosis, atherosclerotic lesions, lowering LDL cholesterol levels, increasing HDL cholesterol levels, increasing reverse cholesterol transport, inhibiting cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, treatment or inhibition of Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer and ihyroiditis, that can downregulate or alter 5-LO gene expression. In this embodiment, the efficacy of a compound administered to a subject for the above-described diseases, disorders or conditions can be tested by isolating a population of cells from the subject, where the population of cells are known to naturally express 5-LO. The population of cells can be isolated before and after treatment of a compound, including after each administration of the compound if the dosing regime comprises multiple and repeated administrations. RNA can be isolated from the cells, such that 5-LO mRNA levels are compared in the samples obtained from before and after compound administration. If the 5-LO mRNA levels are decreased after compound administration, then this indicates that the compound is efficacious. Optimum dosing regimes can in this manner be determined by examining which regime provides the desired decrease in 5-LO gene expression.

The cells that are isolated from the patient can be isolated from tissue biopsies, blood or serum, for example. Specific cell or tissue populations that can be examined include, but are not limited to, platelets, myeloid cells, leukocytes, neutrophils, granulocytes, eosinophils, natural killer cells, T-cells, B-cells, dendritic cells, epidermal cells, Langerhans cells, keratinocytes, glial cells, macrophages, monocytes, mast cells, pulmonary artery endothelial cells, intestinal epithelial cells, vascular tissue, neural tissue, lung tissue, heart tissue, aorta tissue, coronary artery tissue, carotid artery tissue, renal tissue, pineal gland tissue, cerebral cortex tissue; hippocampus tissue, cerebellum tissue, ischemic flap tissue, and tumor tissue.

### EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

### (4-{[3-(3-Benzyl-8-trifluoromethyl-quinolin-4-yl)-phenylamino]-methyl}-phenyl)-acetic acid

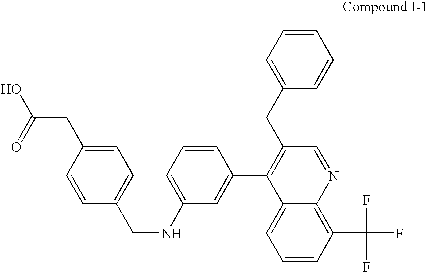

(4-{[3-(3-benzoyl-8-trifluoromethyl-quinolin-4-yl)-phenylamino]-methyl}-phenyl)-acetate was taken up into ethylene glycol along with hydrazine hydrate and heated at 120° C. for 2 hours. Next, a few pellets of KOH were added and the reaction mixture was heated at 180° C. for 4 hours. The reaction mixture was allowed to cool to room temperature, water was added, and the mixture was extracted with ether and concentrated. The resulting material was purified via column chromatography using 5% ethyl acetate in hexane as the eluent to provide Compound I-1. MS (ESI) m/z 527.

### Example 2

### {4-[3-(3-Benzyl-8-trifluoromethyl-quinolin-4-yl)-phenoxymethyl]-phenyl}-acetic acid

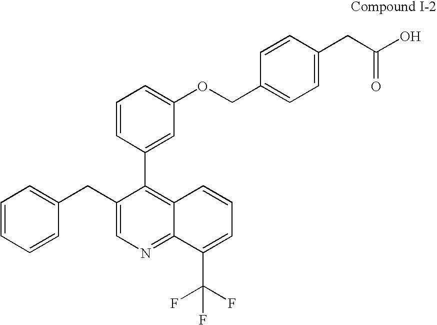

Compound I-2 was prepared as follows. 3-[3-benzyl-8-(trifluoromethyl)quinolin-4-yl]phenol, 4-bromomethyl-phenyl-acetic acid ethyl ester, and K₂CO₃ in acetone was heated to reflux. After 2 hr, the reaction was cooled, filtered, and concentrated. The resulting oil was taken up into THF/MeOH and 2N NaOH was added and the reaction was refluxed. After 2 hr, the reaction was cooled, poured into 2N HCL and extracted with EtOAc. The EtOAc was dried, concentrated, and the product was purified by column chromatography (eluent 40% EtOAc/hexane) to give compound I-2 as a foam. MS (ESI) m/z 528.

### Example 3

### {4-[3-(3-Phenyl-8-trifluoromethyl-quinolin-4-yl)-phenoxymethyl]-phenyl}-acetic acid

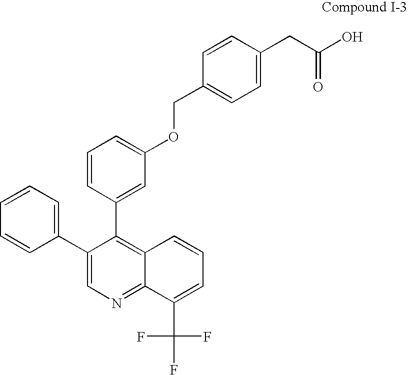

A solution of 3-[3-phenyl-8-(trifluoromethyl)quinolin-4-yl]phenyl acetic acid ethyl ester (0.051 g, 0.10 mmol), and 2N aq NaOH (0.100 mL, 0.20 mmol), in 1:1 ethanol:THF was refluxed at 120° C. for 1 h, cooled and poured into 2N aq. HCl. The solution was extracted with ethyl acetate. The combined extracts were washed with saturated aq. NaHCO₃, water, brine, and dried with magnesium sulfate. The extracts were concentrated and the residue was chromatographed with 1:9 ethyl acetate:hexanes to afford compound I-3 as a colorless solid (0.045 g, 97%); mp 122° C.; MS (ES) m/z 514.2; HRMS: calcd C₃₁H₂₂NF₃O₃+H, 514.16300; found (ESI, [M+H]+), 514.1629.

### Example 4

### {4-[3-(3-Methyl-8-trifluoromethyl-quinolin-4-yl)-phenoxymethyl]-phenyl}-acetic acid

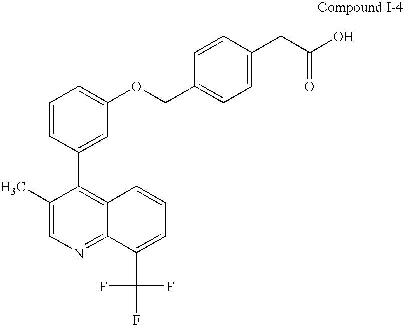

3-[3-Methyl-8-(trifluoromethyl)quinolin-4-yl]phenol (1.13 g, 3.73 mmol) and 4-bromomethylphenylacetic acid (1.02 g, 4.47 mmol) in CH₂Cl₂ (60 mL) was treated with Cs₂CO₃ (4.86 g, 14.9 mmol) and the reaction was stirred overnight. The solvent was removed and the residue dissolved in THF and 2N aq NaOH. After heating at 70° C. for 1 h, the reaction was cooled and the layers separated. The aqueous layer was further extracted with ethyl acetate. The combined organics were dried (Na₂SO₄) and concentrated. The residue was chromatographed eluting with 20:80 ethyl acetate:hexane and then 5:95 methanol:methylene chloride to provide Compound I-4. HRMS: calcd for C₂₆H₂₀F₃NO₃+H, 452.14735; found (ESI, [M+H]+), 452.1452.

### Example 5

### 2-{4-[3-(3-Benzyl-8-trifluoromethyl-quinolin-4-yl)-phenoxymethyl]-phenyl}-2-methyl-propionic acid

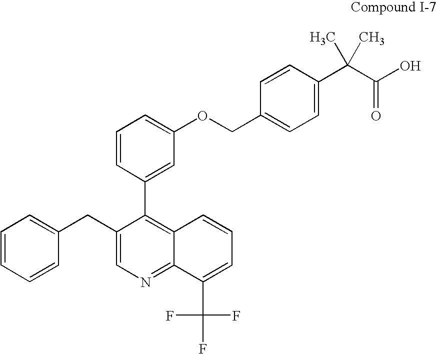

A solution of 3-[3-benzyl-8-(trifluoromethyl)quinolin-4-yl]phenol, 2-(4-bromomethyl-phenyl)-2-methyl-propionic acid methyl ester, and K₂CO₃ in acetone (25 ml) was heated to reflux. After 2 hr, the reaction was cooled, filtered and concentrated. The resulting oil was taken up into THF/MeOH and 2N NaOH was added and the reaction was refluxed. After 2 hr, the reaction was cooled, poured into 2N HCl and extracted with EtOAc. The EtOAc was dried concentrated and the product was purified by column chromatography (eluent 40% EtOAc/ hexane) to give compound I-7 as a foam. MS (ESI) m/z 556.

### Example 6

### 4-[3-(3-Benzyl-8-trifluoromethyl-quinolin-4-yl)-phenoxy]-phenol

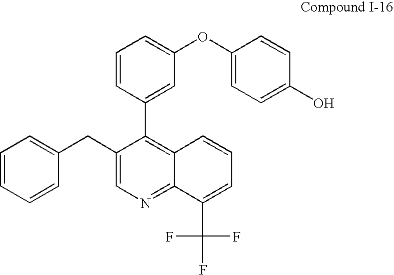

3-Benzyl-4-[3-(4-methoxyphenoxy)phenyl]-8-(trifluoromethyl)quinoline (0.22 g, 0.00045 mol), and Py-HCl was placed into a round bottom flask with a stir bar and lowered into a heating bath at 190-200° C. for 2-3 hr. The vessel was then cooled to RT and 1 N HCl was added to dissolve all solids. The mixture was extracted with EtOAc. The EtOAc layer was washed with water, brine and dried over Na₂SO₄, filtered and concentrated in vacuo to provide a semi-solid material. This material was repeatedly triturated with CH₂Cl₂. The methylene chloride was then concentrated providing the desired crude compound. The crude product was purified by flash chromatography to give compound I-16: mp 55-60° C.; MS (ES) m/z 469.9; HRMS: calcd for C₂₉H₂₀F₃NO₂+H+, 472.15189; found (ESI, [M+H]+), 472.153.

### Example 7

### 4-{[3-(3-Benzyl-8-trifluoromethyl-quinolin-4-yl)-phenylamino]-methyl}-2-methoxy-phenol

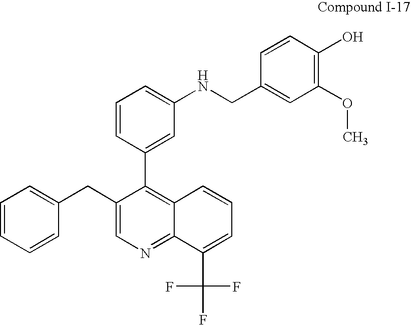

3-(3-Benzyl-8-trifluoromethyl-quinolin-4-yl)-phenylamine and 4-hydroxy-3-methoxy-benzaldehyde were mixed in THF and then treated with NaBH(OAc)₃ and acetic acid. After stirring at 40° C. under an N₂ atmosphere for 2 h the mixture was quenched with water and then extracted with ethyl acetate. The organic residue was purified by silica gel chromatography using 5-50% EtOAc/hexanes as eluent to provide compound I-17. MS (ESI) m/z 515.

### Example 8

### 4-{[3-(3-Benzyl-8-trifluoromethyl-quinolin-4-yl)-phenylamino]-methyl}-2-ethoxy-phenol

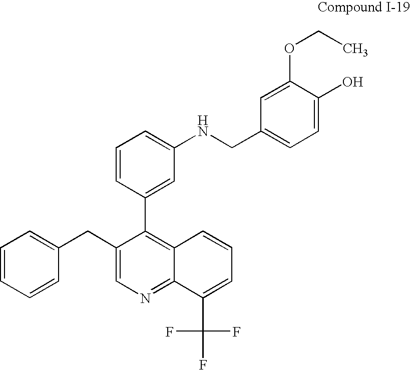

4-{[3-(3-Benzyl-8-(trifluoromethyl)quinolin-4-yl]phenyl}amine and 5-ethoxy-4-hydroxybenzaldehyde were mixed in THF and then treated with NaBH(OAc)₃ and acetic acid. After stirring at 40° C. under an N₂ atmosphere for 2 h the mixture was quenched with water and then extracted with ethyl acetate. The organic residue was purified by silica gel chromatography using 5-50% EtOAc/hexanes as eluent to provide compound I-19. MS (ESI) m/z 529; MS (ESI) m/z 527.

### Example 9

### Method for Identifying or Testing 5-LO Modulators

A panel of LXR or PPAR modulator compounds was tested to determine whether they could downregulate 5-LO gene expression. The compounds were individually tested to determine whether they could downregulate 5-LO gene expression in human and mouse macrophage cell lines.

Preparation of Acetylated LDL: LDL was dialyzed with 500 mL phosphate buffered saline (PBS) three times. 2.5 mL of dialyzed LDL was mixed with 2.5 mL of saturated sodium acetate with continuous stirring in an ice-water bath. Acetic anhydride was added in 7 times 7.5 μL over a period of 1 hour with continuous stirring. Then, the mixture was stirred for an additional 1 hour. The solution was dialyzed against 500 mL PBS containing 0.3 mM EDTA three times. The protein concentration is determined using Bio-Rad DC protein assay kit.

Cell Culture and Compound Treatment: Human THP-1 cells were maintained in RPMI 1640 medium containing 10% Fetal Bovine Serum (FBS), 25 mM Hepes buffer, 50 pg/mL Genamicin, 1 mM Sodium pyruvate, and 50 μM β-mercaptoethanol. THP-1 cells were differentiated with 75 ng/mL phorbal 12,13-dibutyrate (PDBu) three days prior to assay to induce differentiation of the cells into adherent macrophages. Cells were seeded in 12 well plates in 1 mL of complete media. Each well contained 400,000 cells. The cells were stimulated with RPMI 1640 medium (1% FBS) containing 100 μg/mL acetylated-LDL in presence of phorbol ester for 48 hours, including several wells without acetylated-LDL treatment as a control. The medium was then changed to RPMI 1640 medium without FBS, plus the test compounds with phorbol ester for 48 hours. Control cells were not incubated with test compounds. The cells were incubated at 37° C. in a humidified CO₂ incubator. The cells were then ready for RNA extraction.

Murine J774 cells were maintained in DMEM (high glucose) containing 10% FBS, 100 units/mL penicillin, and 100 μg/mL streptomycin. Six hours prior to assay, J774 cells were seeded into 96-well plates in 150 μL complete media. Each well contains 75,000 cells. The cells were stimulated with DMEM-high glucose (1% FBS) containing 100 μg/mL acetylated-LDL for 24 to 48 hours, including several wells without acetylated-LDL treatment as a control. The media was then changed to DMEM-high glucose (without FBS) plus the test compounds for 24 to 48 hours. Compounds were dosed in DMSO vehicle and control cells were treated with vehicle alone. The cells were incubated at 37° C. in a humidified CO₂ incubator. The cells were then ready for RNA isolation.

RNA analysis: Total RNA was isolated from the THP-1 cells by using a QIAGEN RNeasy Mini Kit. Real-time quantitative RT-PCR assays were performed using an Applied Biosystems 7900 sequence detector. Each amplification mixture (50 μL) contained 50-100 ng of total RNA, 200 nM forward primer, 200 nM reverse primer, and 200 nM dual-labeled fluorogenic probe. The RT-PCR reagent is from QIAGEN (QuantiTect Probe RT-PCR kit). The RT-PCR thermocycling parameters were 48° C. for 30 minutes, 95° C. for 10 minutes, and 40 cycles at 95° C. for 15 seconds and 55° C. for 1 minute. The samples, no-RT controls, and serially diluted RNA standards were analyzed in parallel. The 5-LO target gene was analyzed and normalized for human glyceraldehyde-3-phosphate dehydrogenase (GAPDH) expression using probe and primers from predeveloped assays for GAPDH (Applied Biosystems). Quantitative analysis was performed using the threshold procedure, and relative amounts were calculated from the standard curve.

Total RNA from the J774 cells was isolated by using the ABI 6100 RNA isolation system for 96-well plates (the THP-1 cells can also be cultured and tested in 96-well plates). Real-time quantitative RT-PCR assays were performed using an Applied Biosystems 7900 sequence detector. Each amplification mixture (50 μL) contained 50-100 ng of total RNA, 200 nM forward primer, 200 nM reverse primer, and 250 nM dual-labeled fluorogenic probe. The RT-PCR reagent is from QIAGEN (QuantiTect Probe RT-PCR kit). The RT-PCR thermocycling parameters were 48° C. for 30 minutes, 95° C. for 10 minutes, and 40 cycles at 95° C. for 15 seconds and 60° C. for 1 minute. The samples, no-RT controls, and serially diluted RNA standards were analyzed in parallel. The 5-LO target gene was analyzed and normalized for 18S ribosomal RNA expression using probe and primers from predeveloped assays for 18S (Applied Biosystems). Quantitative analysis was performed using the threshold procedure, and relative amounts were calculated from the standard curve.

Primers for RT-PCR can be designed according to procedures known to one skilled in the art. The primers are designed in view of the 5-LO mRNA sequence, which can be obtained from Genbank or the literature. For example, the Genbank accession number for the human 5-LO mRNA message is NM 00698; see also Dixon, R. A. et al., Proc. Natl. Acad. Sci. U.S.A. 85 (2), 416-420 (1988); Matsumoto, T. et al., Proc. Natl. Acad. Sci. U.S.A. 85 (1), 26-30 (1988). The Genbank accession number for the murine 5-LO mRNA message is L42198, see also Chen, X. S. et al., J. Biol. Chem. 270 (30), 17993-17999 (1995).

Twenty-three exemplary 5-LO modulator compounds of the present invention were able to inhibit or reduce 5-LO gene expression, as reflected by the decrease in 5-LO mRNA message in the total RNA. The results for the twenty-three (23) compounds are summarized in the table below:

| | % Inhibition | | | |
|---|---|---|---|---|
| | of 5-LO | % Inhibition of | % Inhibition of | % Inhibition of |
| | mRNA at | 5-LO mRNA at | 5-LO mRNA at | 5-LO mRNA at |
| Com- | 0.1 uM of | 0.3 uM of | 1.0 uM of | 3.0 uM of |
| pound | Compound | Compound | Compound | Compound |
| I-1 | 91 | 82 | 88 | 83 |
| I-2 | 84 | N/A | 92 | N/A |
| I-3 | 88 | N/A | 89 | N/A |
| I-4 | 79 | N/A | 90 | N/A |
| I-5 | 87 | N/A | 91 | N/A |
| I-6 | 62 | N/A | 81 | N/A |
| I-7 | 87 | N/A | 88 | N/A |
| I-8 | 89 | N/A | 93 | N/A |
| I-9 | 22 | N/A | 45 | N/A |
| I-10 | 86 | N/A | 83 | N/A |
| I-11 | N/A | 74 | N/A | 71 |
| I-12 | N/A | 71 | N/A | 76 |
| I-13 | N/A | 40 | N/A | 30 |
| I-14 | N/A | 81 | N/A | 84 |
| I-15 | N/A | 79 | N/A | 76 |
| I-16 | N/A | 60 | N/A | 61 |
| I-17 | N/A | 41 | N/A | 53 |
| I-18 | N/A | 63 | N/A | 58 |
| I-19 | N/A | 22 | N/A | 60 |
| I-20 | N/A | 78 | N/A | 78 |
| I-21 | N/A | 78 | N/A | 63 |
| I-22 | N/A | 67 | N/A | 55 |
| I-23 | N/A | 76 | N/A | 92 |

*N/A or not-allowed refers to the fact that the compound was not tested at the amount in question.

What is claimed:

1. A method for downregulating 5-lipoxygenase gene expression comprising contacting a cell or a tissue with a compound of Formula I 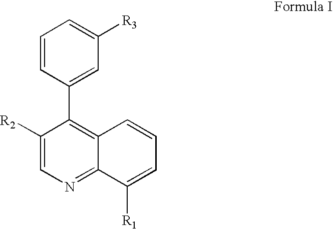
wherein
R₁ is —CF₃ or —Cl;
R₂ is —CH₃, 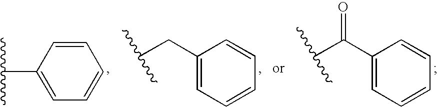
and
R₃ is 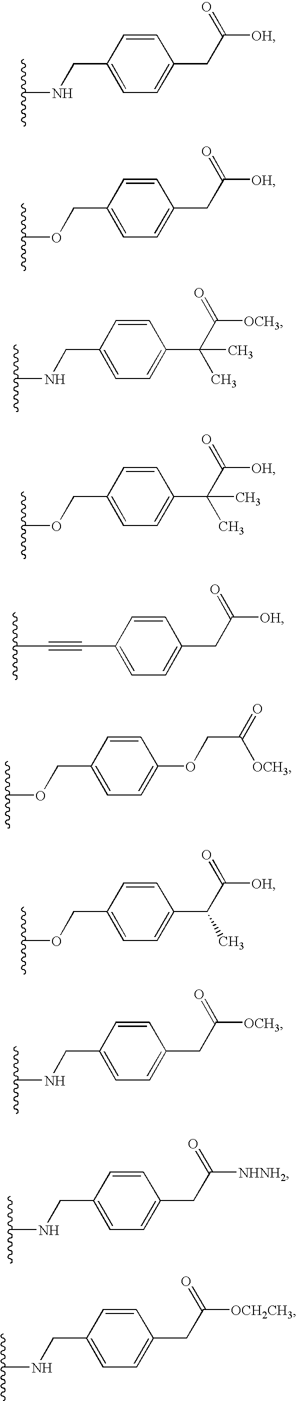 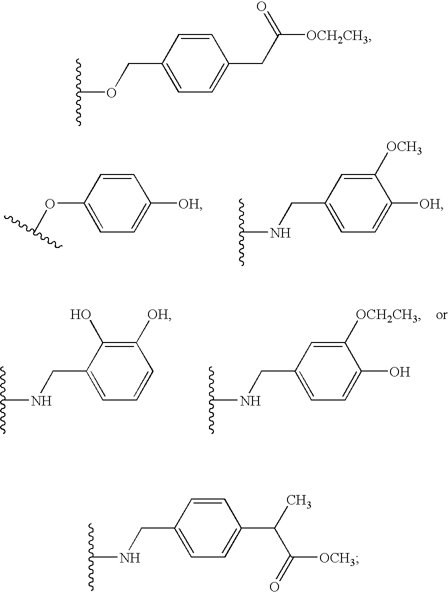
or a pharmaceutically acceptable salt thereof.

2. The method of claim 1, wherein the cell or tissue comprises a platelet, a myeloid cell, a leukocyte, a neutrophil, a granulocyte, an eosinophil, a natural killer cell, a T-cell, a B-cell, a dendritic cell, an epidermal cell, a Langerhans cell, a keratinocyte, a glial cell, a macrophage, a monocyte, a mast cell, a pulmonary artery endothelial cell, an intestinal epithelial cell, vascular tissue, neural tissue, lung tissue, heart tissue, cardiovascular tissue, aorta tissue, coronary artery tissue, carotid artery tissue, renal tissue, pineal gland tissue, cerebral cortex tissue, hippocampus tissue, cerebellum tissue, ischemic flap tissue, pancreatic tissue or tumor tissue.

3. A method for treating a condition, disease or disorder involving leukotriene-mediated inflammation or leukotriene-mediated cell signaling in a subject, the method comprising administering to the subject an effective amount of a compound of Formula I 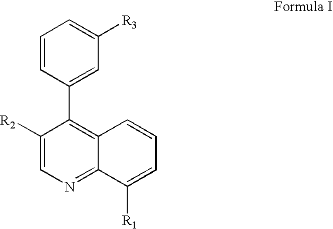
wherein
R₁ is —CF₃ or —Cl;
R₂ is —CH₃, 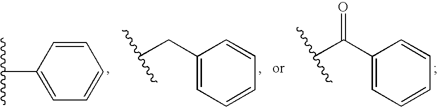
and
R₃ is 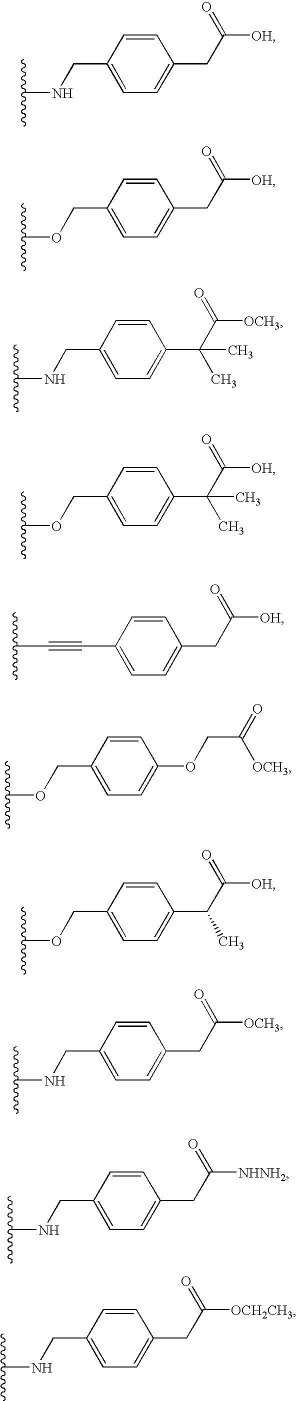 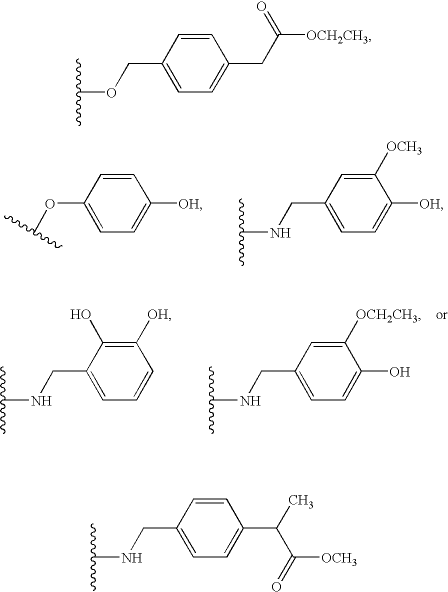
or a pharmaceutically acceptable salt thereof.

4. The method of claim 3, wherein the subject suffers from a disease, disorder or condition comprising atherosclerosis, atherosclerotic lesions, high LDL cholesterol levels, low HDL cholesterol levels, abnormal reverse cholesterol transport, abnormal cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer or thyroiditis.

5. The method of claim 4, wherein the disease, disorder or condition comprises vascular dysfunction, hypertension, acute coronary syndrome, disorders of triglyceride metabolism, metabolic syndromes, Syndrome X, disorders in leukotriene synthesis, asthma, Sjogren-Larsson syndrome (SLS), stroke, seizure, prion disease, aging-associated neurodegeneration, or cancer.

6. A method for reducing leukotriene synthesis in a subject, the method comprising administering to the subject an effective amount of a compound of Formula I 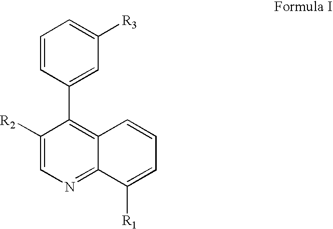
wherein
R₁ is —CF₃ or —Cl;
R₂ is —CH₃, 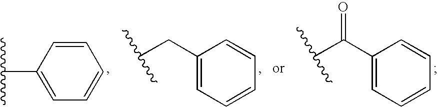
and
R₃ is 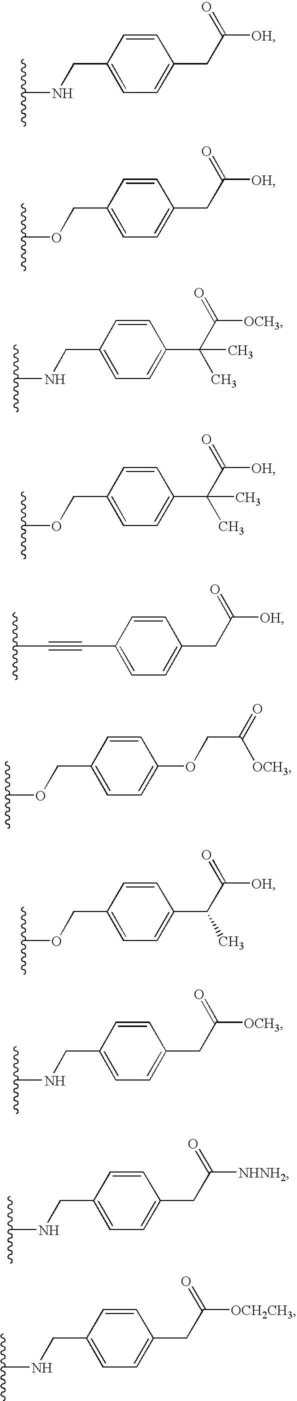 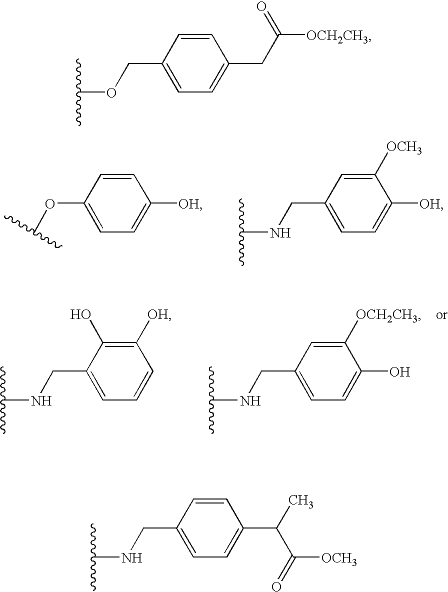
or a pharmaceutically acceptable salt thereof.

7. A method for treating inflammation in a subject, the method comprising administering to the subject an effective amount of a compound of Formula I 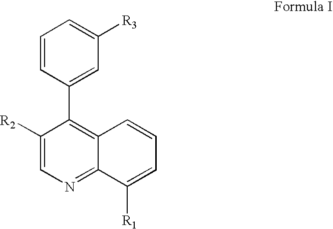
wherein
R₁ is —CF₃ or —Cl;
R₂ is —CH₃, 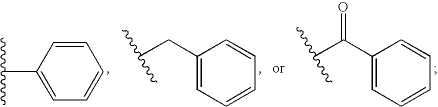
and
R₃ is 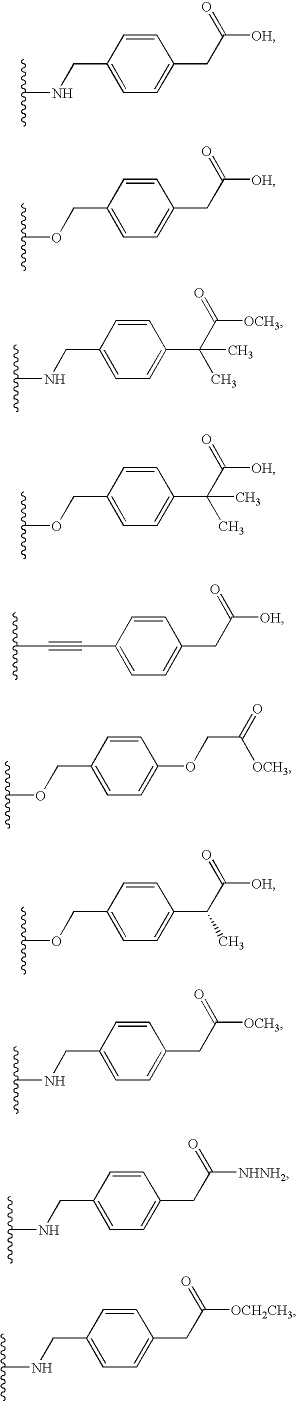 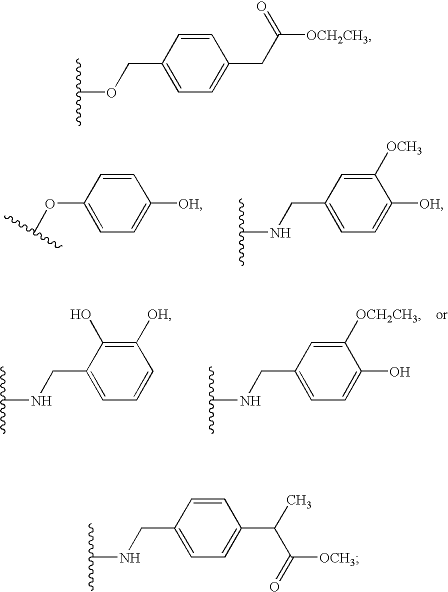
or a pharmaceutically acceptable salt thereof.

8. A method for treating atherosclerosis in a subject, the method comprising administering to the subject an effective amount of a compound of Formula I 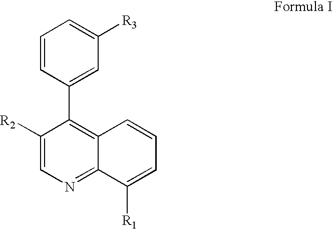
wherein
R₁ is —CF₃ or —Cl;
R₂ is —CH₃, 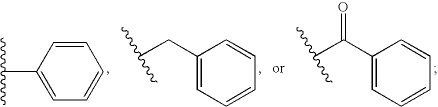
and
R₃ is 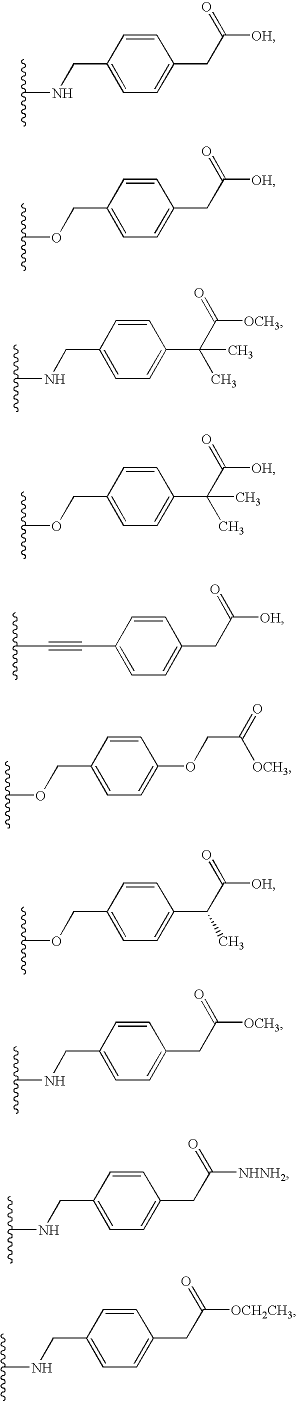 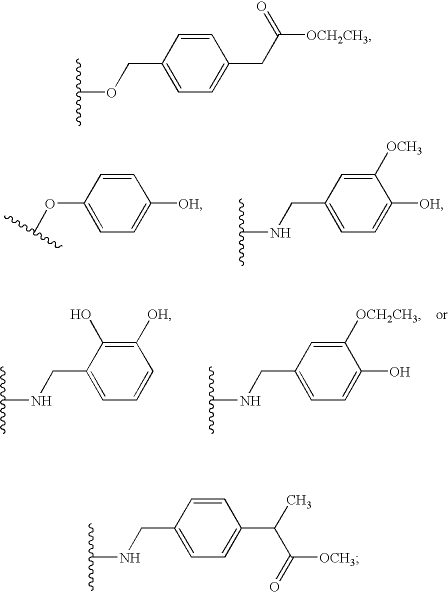
or a pharmaceutically acceptable salt thereof.

9. A method for screening a compound to be a candidate for treating conditions, diseases or disorders involving leukotriene-mediated inflammation or leukotriene-mediated cell signaling, the method comprising:
(a) contacting a cell with the compound; and
(b) determining whether 5-lipoxygenase gene expression is decreased in the cell of step (a) as compared to a cell that has not been contacted with the compound, wherein if 5-lipoxygenase gene expression is decreased in the cell of step (a), then the compound is a candidate for treating conditions, diseases or disorders involving leukotriene-mediated inflammation or leukotriene-mediated cell signaling.

10. A method for screening a compound to be a candidate for treating conditions, diseases or disorders involving leukotriene-mediated inflammation or 5-lipoxygenase mediated lipid oxidation, the method comprising:
(a) activating a macrophage cell with acetylated-LDL;
(b) contacting the macrophage cell with the compound;
(c) determining whether 5-lipoxygenase gene expression is decreased in the macrophage cell of step (b) as compared to a macrophage cell that has not been contacted with the compound, wherein if 5-lipoxygenase gene expression is decreased in the macrophage cell of step (b), then the compound is a candidate for downregulating 5-lipoxygenase gene expression.

11. The method of claim 9 or 10, wherein the compound is a Liver X Receptor (LXR) modulator compound.

12. The method of claim 11, wherein the compound comprises a quinoline.

13. The method of claim 9 or 10, wherein the compound is a Peroxisome Proliferator Activated Receptor (PPAR) modulator compound.

14. The method of claim 12, wherein the compound comprises a quinoline.

15. The method of claim 9, wherein the conditions, diseases or disorders involving leukotriene-mediated inflammation or leukotriene-mediated cell signaling comprises atherosclerosis, atherosclerotic lesions, high LDL cholesterol levels, low HDL cholesterol levels, abnormal reverse cholesterol transport, abnormal cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer or thyroiditis.

16. The method of claim 10; wherein the conditions, diseases or disorders involving leukotriene-mediated inflammation or 5-lipoxygenase mediated lipid oxidation comprise atherosclerosis, atherosclerotic lesions, high LDL cholesterol levels, low HDL cholesterol levels, abnormal reverse cholesterol transport, abnormal cholesterol absorption, vascular dysfunction, hypertension, acute coronary syndrome, disorders of triglyceride metabolism, metabolic syndromes, Syndrome X, diabetes, type I diabetes, type II diabetes, insulin resistance, inflammation, autoimmune disease, arthritis, rheumatoid arthritis, disorders in leukotriene synthesis, asthma, Alzheimer's disease, Sjogren-Larsson syndrome (SLS), stroke, seizure, prion disease, aging-associated neurodegeneration, multiple sclerosis, restenosis, inflammatory bowel disease (IBD), Crohn's disease, endometriosis, celiac, cancer, lung cancer or thyroiditis.

17. A method for assessing or testing the efficacy of a 5-lipoxygenase modulator compound that has been administered to a subject, the method comprising
(a) isolating a first cellular sample from the subject;
(b) administering to the subject an amount of a compound of Formula I 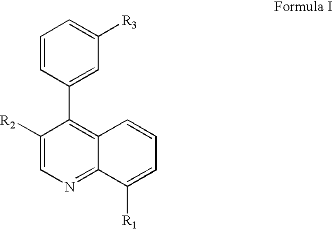
wherein
R₁ is —CF₃ or —Cl;
R₂ is —CH₃, 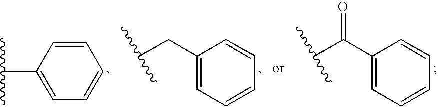
and
R₃ is 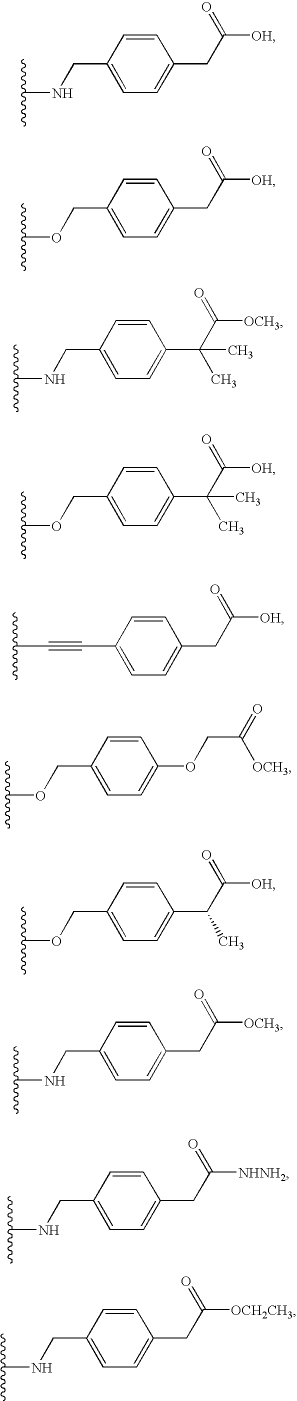 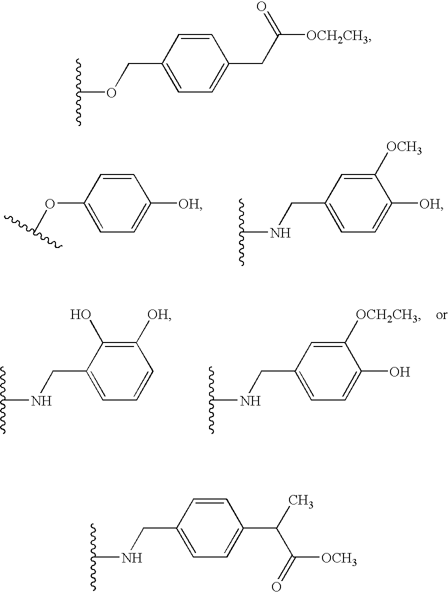
or a pharmaceutically acceptable salt thereof;
(c) isolating a second cellular sample from the subject after administration of the compound; and
(d) determining whether 5-lipoxygenase gene expression is reduced in the second cellular sample as compared to the first cellular sample, wherein if 5-lipoxygenase gene expression is reduced, then the compound has been administered in an effective amount.

18. The method of claim 17, further comprising:
(e) at least one additional administration of the compound or a pharmaceutically acceptable salt thereof;
(f) at least one additional isolation of a cellular sample from the subject subsequent to the additional administration(s) in step (e); and
(g) at least one additional determination of whether 5-lipoxygenase gene expression is reduced in the additional cellular sample(s) as compared to the first cellular sample, wherein if 5-lipoxygenase gene expression is reduced, then the compound has been administered in an effective amount.

19. A pharmaceutical kit comprising a unit dosage form of a compound having the Formula I 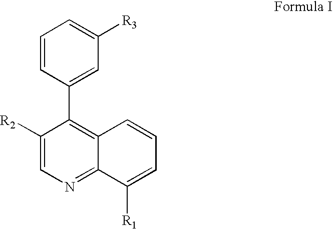
wherein
R₁ is —CF₃ or —Cl;
R₂ is —CH₃, 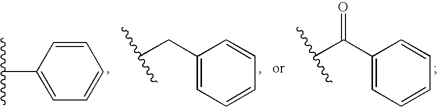
and
R₃ is 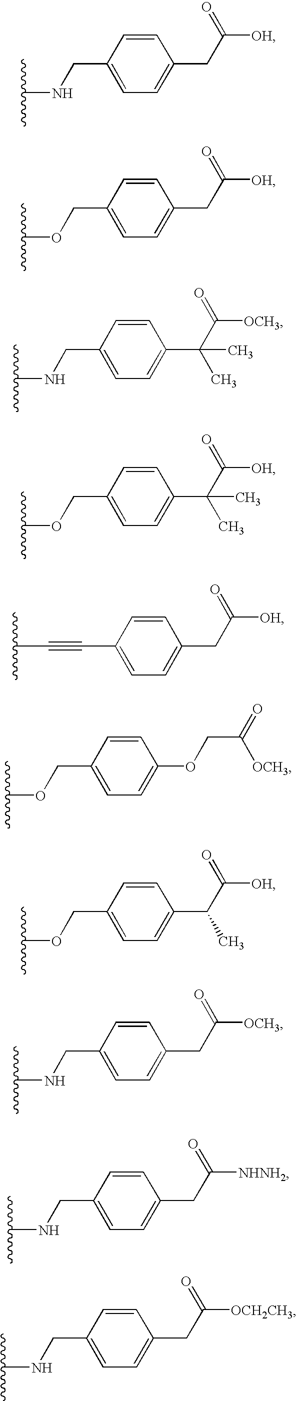 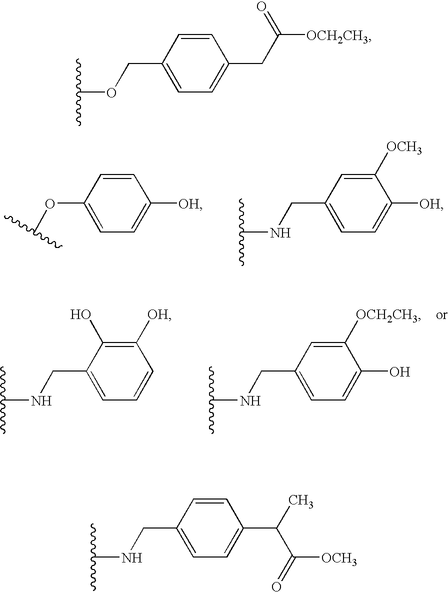
or a pharmaceutically acceptable salt thereof.

20. The kit of claim 19, wherein the unit dosage form comprises a container containing an effective amount of the compound and a physiologically acceptable carrier or vehicle.

21. The kit of claim 20, further comprising a label or printed instructions instructing the use of the compound to treat or prevent a condition.

22. The kit of claim 20, wherein the unit dosage form further comprises an effective amount of another therapeutic agent.
